(19) United States
(12) Patent Application Publication
(10) Pub. No.: US 2008/0039415 A1
(43) Pub. Date: Feb. 14, 2008

(54) RETROGRADE TRANSPORT OF SIRNA AND THERAPEUTIC USES TO TREAT NEUROLOGIC DISORDERS

(76) Inventors: Gregory Robert Stewart, Plymouth, MN (US); Dinah Sah, Boston, MA (US)

Correspondence Address: FOX ROTHSCHILD LLP;PRINCETON PIKE CORPORATE CENTER, 997 LENOX DRIVE, BUILDING #3, LAWRENCEVILLE, NJ, 08648, US

(21) Appl. No.: 11/464,074

(22) Filed: Aug. 11, 2006

(51) Int. Cl. A61K 48/00

(52) U.S. Cl. 514/44

(57) ABSTRACT

Methods of treating disorders affecting the central nervous system (CNS) are disclosed. More particularly, methods of treating neurological disorders are disclosed which show therapeutic or prophylactic treatment of a mammalian CNS disorder by effecting local administration of an iRNA agent, followed by retrograde transport of the iRNA agent away from the administration site and onto multiple regions within the CNS. This retrograde transport of iRNA results in an improved therapeutic involvement for the respective iRNA agent.

### FIELD OF THE INVENTION

The present invention relates to methods of treating disorders affecting the central nervous system (CNS), and more particularly to methods of treating CNS disorders whereby the iRNA agent undergoes retrograde transport away from a local administration site to impart an improved therapeutic or prophylactic biological effect.

### BACKGROUND OF THE INVENTION

RNA interference or “RNAi” is a term initially coined by Fire and co-workers to describe the observation that double-stranded RNA (dsRNA) can block gene expression when it is introduced into worms (Fire et al., Nature 391:806-811, 1998). Short dsRNA directs gene-specific, post-transcriptional silencing in many organisms, including vertebrates, and has provided a new tool for studying gene function. RNAi also has great therapeutic potential by the manufacture of synthetic inhibitory RNA (iRNA) that selectively target and disrupt the mRNA transcription product of a particular gene leading to suppression of protein expression. Within the context of neurology, there are numerous diseases that could be treated based on targeted suppression of a particular gene product including, without limitations, Alzheimer's disease, Parkinson's disease, Motor Neuron Disease including Amyotrophic Lateral Sclerosis, Metabolic Storage disease, neuropathies and Huntington's disease. The latter is an example of a (CNS) disorder that results from an the dominantly inherited expansion of nucleotide repeats within genomic DNA, including, without limitations, Huntington's disease (HD), spinocerebellar ataxia (SPA 1, 2, 3, 6, 7, and 17), dentarubral-pallidoluysian atrophy (DRPLA), spinobulbar muscular atrophy (SBMA), and myotonic dystrophy (DM1 and DM2). Such disorders are prime candidates for iRNA therapy because a specific gene and protein product have been identified as causing the disease. Huntington's disease (HD) is an autosomal dominant neurodegenerative disease that is characterized by involuntary movement, dementia and behavioral changes. The underlying cause of HD is a gain of function mutation in the HD gene (htt). Therefore, it is plausible that suppressing htt activity may provide for an effective treatment for this disorder. The htt mutation is characterized by multiple trinucleotide CAG repeats within the gene. Normal htt alleles comprise 26 or fewer CAG repeats, with intermediate alleles containing from about 27-35 CAG repeats. Alleles with CAG repeats above 36 are associated with HD individuals. Beyond this number, the greater the number of repeats the more likely the chance of developing HD symptoms, and for such symptoms to occur at a younger age. Symptoms include a progressive loss of mental function, including personality changes, and loss of cognitive functions such as judgment, and speech. To date there is no effective treatment for HD. To this end, there remains a need to develop an effective therapy for CNS-based dominantly inherited nucleotide repeat diseases, including but not limited to Huntington's disease. The current state of the art regarding iRNA technology and relating to possibly treating CNS-based dominantly inherited nucleotide repeat diseases is reviewed by Denovan-Wright and Davidson (2006, Gene Therapy 13:525-531).

Due to the presence of the blood brain barrier, iRNA molecules will not enter the brain from the blood. In order to treat a neurologic disorder, such as HD, iRNA must be directly injected into the brain. This can be readily accomplished with placement of a catheter into the brain parenchyma targeting a specific region or structure. However, it is well known that distribution of any agent injected into the parenchyma, particularly large molecules, is very limited. As with many neurologic disorders, HD affects multiple different, but interconnected brain regions each requiring therapeutic delivery of iRNA for treatment. It is neither practical, feasible or safe to contimplate multiple injection into the brain, particularly on a chronic basis as would be needed for iRNA therapy. Therefore, the ability to effectively treat a neurologic disorder with iRNA is compromised by an inability to effectively distribute iRNA within and across multiple brain regions. The present invention addresses and meets this need by disclosing a method of treating such neurological disorders which comprises administering a gene-specific iRNA agent to an afflicted or at risk subject and having the iRNA agent transported in a retrograde manner away from the site of administration so as to impart an improved biological effect.

### SUMMARY OF THE INVENTION

The present invention relates to a method of therapeutic or prophylactic treatment of a mammalian CNS disorder by effecting local administration of an iRNA agent which is accompanied by subsequent retrograde transport of the iRNA agent to multiple regions within the CNS. The retrograde transport away from the local region of iRNA administration results in an improved therapeutic involvement for the respective iRNA agent. Therefore, methods of treatment are provided herein which rely on local delivery of an iRNA agent and subsequent retrograde transport of that iRNA agent to other regions of the CNS. These methods provide for delivery and retrograde transport of iRNA agents within neurons to prevent and/or treat neurological diseases.

To that end, the present invention relates to a method of treating a central nervous system disorder in a mammal (e.g., a human) which comprises administering or contacting a RNA agent or iRNA agent to a neuron at a first site in the central nervous system and having the RNA agent undergo retrograde transport from the first site to one or more secondary sites within the central nervous system to impart a therapeutic effect at CNS regions away from the first site of administration. Retrograde transport to these secondary sites may involve retrograde transport to one or more secondary sites away from the first site and may include the ability to impart a measurable therapeutic effect for a range of distances away from the local/first site of administration, including but not limited to distances of at least 2 mm from the site of administration. A person of ordinary skill in the art would understand that the iRNA agent of the present invention can be retrogradely transported to a secondary site which may be far removed from the first site, for example, in an embodiment wherein the iRNA agent is delivered to the axons of the cells projecting from brain to spinal cord or wherein the iRNA agent is delivered to the axons of the motor neurons projecting to toes or feet.

In another aspect, the present invention relates to methods of treating a central nervous system disorder in a human by contacting an iRNA agent which undergoes retrograde transport away from the local site of administration, as described herein, wherein the central nervous system disorder is a dominantly inherited nucleotide repeat disorder, including but not limited to Huntington's disease (HD), spinocerebellar ataxia (SPA 1, 2, 3, 6, 7, and 17), dentarubral-pallidoluysian atrophy (DRPLA), spinobulbar muscular atrophy (SBMA), and myotonic dystrophy (DM1 and DM2). An exemplified embodiment of the this portion of the invention relates to a method of treating Huntington's disease (HD) via local CNS administration of particular iRNA agents which target the huntingtin (htt) gene, where it is shown that these iRNAs undergo retrograde transport to CNS regions distinct from the local site of iRNA administration. To this end, the present invention relates to methods of prophylactic and/or therapeutic treatment of CNS disorders by effecting widespread, retrograde distribution of siRNAs targeting the htt gene in the CNS following chronic intrastriatal infusion. Subsequent to local administration by intrastriatal infusion, the respective htt iRNA undergoes retrograde transport distally, contralaterally or ipsilaterally to the administration site at a therapeutic level at least 2, 3, 5, 8, 10, 15, 20, 25, 30, 35, 40, 45, or 50 mm, being taken up by neurons with processes or endings at or near the administration site and whose cell bodies are located in such regions as the cortex, thalamus, substantia nigra of the central nervous system, or any combination thereof. iRNA agents from Table 1 are provided as examples, and are not meant to denote any sort of limitation to the array of iRNA agents that may be useful to practice methods of down regulating htt gene expression. Intrastriatal infusion over a given time period may be utilized to deliver an iRNA agent for applying a therapeutic treatment to any of the CNS disorders contemplated in the present invention. For example a pump implanted under the skin with interconnected catheter placed in the brain can be used to deliver iRNA on a chronic basis for months to years.

The treatment methods of the present invention rely on iRNA agents which are optimized for neuronal uptake and/or increased stability at and around the site of local administration. As discussed herein, such iRNA agents may be in the form of a double stranded RNA duplex and/or may contain modifications to promote such cell uptake and/or iRNA stability, such as inclusion of lipophilic moiety, such as a cholesterol moiety.

As used herein, “retrograde transfer” or “retrograde transport” is meant to denote the measured ability of targeted RNA agent or iRNA agent to migrate substantially away from the site of local administration along axons or neuronal processes to distal neuronal cell bodies at locations removed from the injection site so as to maximize the therapeutic or prophylactic effect intended by the initial administration of the respective RNA agent or iRNA agent. Such post-administration movement may be in any reasonable manner and is contemplated to involve transfer ranges in the of about at least about 2, 3, 5, 8, 10, 15, 20, 25, 30, 35, 40, 45 or 50 mm from the site of administration.

As used herein, a “neural gene” is a gene expressed in neural cells (e.g., htt). A neural gene can be expressed exclusively in neural cells, or can be expressed in other cell types in addition to the neural cell. In one embodiment, neural gene expression can be evaluated by a method to examine neural RNA levels (e.g., Northern blot analysis, RT-PCR, RNAse protection assay, or branched DNA assay) or neural polypeptide levels (e.g., Western blot, immunohistochemistry, or autofluorescence assays (e.g., to detect GFP or luciferase expression)).

As used herein, a “neural cell” is a cell of the nervous system, e.g., the peripheral or the central nervous system. A neural cell can be a nerve cell (i.e., a neuron), e.g., a sensory neuron or a motor neuron, or a glial cell. Exemplary neurons include dorsal root ganglia of the spinal cord, spinal motor neurons, retinal bipolar cells, cortical and striatal cells of the brain, hippocampal pyramidal cells, and purkinje cells of the cerebellum. Exemplary glial cells include oligodendrocytes and astrocytes of the central nervous system, and the Schwann cells of the peripheral nervous system.

As used herein, “enhanced uptake into neural cells” is meant that higher levels of a modified iRNA agent are incorporated into a neural cell than unmodified iRNA agent when the cells exposed to each type of iRNA agent are treated under similar conditions, in in vitro or in vivo conditions.

As used herein, an “RNA agent” is an unmodified RNA, modified RNA, or nucleoside surrogates, which are described herein or are well known in the RNA synthetic art. While numerous modified RNAs and nucleoside surrogates are described, preferred examples include those which have greater resistance to nuclease degradation than do unmodified RNAs. Preferred examples include those that have a 2′ sugar modification, a modification in a single strand overhang, preferably a 3′ single strand overhang, or, particularly if single stranded, a 5′ modification which includes one or more phosphate groups or one or more analogs of a phosphate group.

As used herein, the terms “iRNA agent” (abbreviation for “interfering RNA agent”) or “siRNA (abbreviation for “small interfering RNA agent”) are used interchangeably to denote an RNA agent, which can downregulate the expression of a target gene, preferably an endogenous or pathogen target RNA expressed in a neural cell, especially a neuron. While not wishing to be bound by theory, an iRNA agent or siRNA may act by one or more of a number of mechanisms, including post-transcriptional cleavage of a target mRNA sometimes referred to in the art as RNAi, or pre-transcriptional or pre-translational mechanisms. An iRNA agent is preferably a double stranded (ds) iRNA agent.

### BRIEF DESCRIPTION OF THE DRAWINGS

FIGS. 1A, 1B, 1C, and 1D show, after intrastriatal pump infusion, Cy3-Htt siRNA distribution in rat brain (#939), demonstrating neuronal uptake that appears to be cytoplasmic. FIG. 1A, Cx=cortex; FIG. 1B, Str=striatum; FIG. 1C, Thal=thalamus; FIG. 1D, SN=substantia nigra.

FIGS. 2A, 2B, 2C, and 2D show, after intrastriatal pump infusion, Cy3-cholesterol-Htt siRNA uptake in white matter fiber bundles in striatum from four different rats. Str=striatum.

FIGS. 3A, 3B, 3C, and 3D show, after intrastriatal pump infusion, Cy3-cholesterol-Htt siRNA uptake in thalamus (FIGS. 3A and 3B) and substantia nigra (FIGS. 3C and 3D) from two different rats. Thal=thalamus, SN=substantia nigra.

FIGS. 4A and 4B show (A) images demonstrating that cortical distribution of Cy3-Htt siRNA does not overlap with GFAP immunostaining (dark brown) in rat striatum; and, (B) images demonstrating that cortical distribution of Cy3-Htt siRNA does not overlap with Iba1 immunostaining (dark brown) in rat striatum.

### DETAILED DESCRIPTION OF THE INVENTION

The present invention relates to methods of prophylactic or therapeutic treatment of CNS disorders by effecting widespread local and subsequent retrograde distribution of RNA agent and/or iRNA agents within the CNS. The methods disclosed herein provide for local delivery and retrograde transport of RNA agents and iRNA agents within neurons to prevent and/or treat neurological diseases. Such methodology relies on local administration of an iRNA agent which is accompanied by subsequent retrograde transport of the iRNA agent to multiple regions within the CNS. The retrograde transport away from the local region of iRNA administration results in an improved therapeutic involvement for the respective iRNA agent. Therefore, methods of treatment are provided herein which rely on local delivery of an iRNA agent and subsequent retrograde transport of that iRNA agent to other regions of the CNS. These methods provide for delivery and retrograde transport of iRNA agents within neurons to prevent and/or treat neurological diseases.

The present invention also relates to methods of prophylactic or therapeutic treatment of CNS disorders by effecting widespread distribution of iRNAs agents targeting the htt gene within the CNS. A person of ordinary skill in the art will appreciate that the methods of the present invention are suitable for treatment of a variety of diseases. Among these diseases are dominantly inherited diseases including, without limitation, Huntington's disease, spinocerebellar ataxia 1, 2, 3, 6, 7, and 17, dentarubral-pallidoluysian atrophy, spinobulbar muscular atrophy, and myotonic dystrophy. In another aspect, the methods of the instant invention are suitable for other diseases. Suitable non-limiting examples of the latter group of diseases include Alzheimer's disease and Parkinson's disease. A person of ordinary skill in the art knows or can easily find the information about the genes involved in the pathogenesis of these disorders, and thus would be able to define gene targets for each of the diseases recited above. In a non-limiting example, an appropriate target gene for Alzheimer's disease is BACE1 (beta-amyloid cleaving enzyme 1, including variants A, B, C, and D, GenBank Accession Numbers NP_{—}036236, NP_{—}620428, NP_{—}620427, and NP_{—}620429, respectively). In another non-limiting example, alpha-synuclein (NP_{—}000336 and NP_{—}009292 for different isoforms) is a promising target for the treatment of Parkinson's disease by an iRNA agent. In yet another non-limiting example, ataxin 1 (NP_{—}000323) is a major factor in pathogenesis of Spinocerebellar Ataxia Type 1.

An exemplified embodiment of the this portion of the invention relates to a method of treating Huntington's disease (HD) via local CNS administration of particular iRNA agents which target the huntingtin (htt) gene, where it is shown that these iRNA agents undergo retrograde transport to CNS regions distinct from the local site of iRNA administration. To this end, the present invention relates to methods of prophylactic and/or therapeutic treatment of CNS disorders by effecting widespread, retrograde distribution of siRNAs targeting the htt gene in the CNS following chronic intrastriatal infusion. Subsequent to local administration by intrastriatal infusion, the respective htt iRNA undergoes retrograde transport distally, contralaterally or ipsilaterally to the administration site at a therapeutic level where the retrograde transport occurs over a distance of at least 2, 3, 5, 8, 10, 15, 20, 25, 30, 35, 40, 45, or 50 mm, being taken up by neurons with processes or endings at or near the administration site and whose cell bodies are located in such regions as the cortex, thalamus, substantia nigra of the central nervous system, or any combination thereof. iRNA agents from Table 1 are provided as examples, and are not meant to denote any sort of limitation to the array of iRNA agents that may be useful to practice methods of down regulating htt gene expression. Intrastriatal infusion over a given time period may be utilized to deliver an iRNA agent for applying a therapeutic treatment to any of the CNS disorders contemplated in the present invention. Huntington's disease (HD) is an autosomal dominant neurodegenerative disease that is characterized by involuntary movement, dementia, and behavioral changes. The underlying cause of HD is a gain of function mutation in the gene encoding huntingtin (htt) and suppression of htt should provide an effective treatment for this disease. To that end, siRNAs are synthetic, double-stranded oligoribonucleotides that harness RNA interference (RNAi), a naturally occurring cellular mechanism for selectively down-regulating gene expression and reducing levels of the corresponding protein. The intracerebral distribution of Cy3-tagged siRNAs that target htt mRNA in the rat brain after continuous 12 day infusion with Alzet osmotic pumps is exemplified. Unconjugated and cholesterol-conjugated siRNAs are compared. Following chronic intrastriatal infusion, bright fluorescent label was present surrounding the injection site and extending into the overlying cortex. Both neuronal cell bodies and fibers were intensely labeled (negative controls included infusion of PBS) Outside of the striatum, discrete cellular labeling was also observed in the substantia nigra pars compacta and thalamus consistent with retrograde transport of siRNA to structures with known projections to the striatum. The distribution of labeled siRNA (local and distant structures) was similar for conjugated and unconjugated forms of siRNA, although the former yielded more discrete labeling of neuronal structures. These results demonstrate that continuous delivery of siRNA to the striatum distributes both locally and distally to brain structures relevant to the treatment of HD and other neurodegenerative disorders. To this end, one aspect of the invention relates to a method of treating or preventing a neurological disorder which features a method of treating a subject having, or at risk for developing a neurological disorder by administering an iRNA agent that inhibits expression of a gene expressed in neurons. In one embodiment, the iRNA agent modified for enhanced uptake into neurons can inhibit, or decrease, expression of the huntingtin (htt) gene in a human having or at risk for developing Huntington's Disease (HD).

In a typical embodiment, the subject or host is a mammal such as a cow, horse, mouse, rat, dog, pig, goat, or a primate. The subject can be a dairy mammal (e.g., a cow, or goat) or other farmed animal (e.g., a chicken, turkey, sheep, pig, fish). However, a preferred embodiment for practicing the methods disclosed herein is where the subject is a human, e.g., a normal individual or an individual that has, is diagnosed with, or is predicted to have a neurological disease or disorder, including but not limited to Huntington's disease.

To this end, the present invention relates to the administration of an iRNA to the CNS of a host followed by the retrograde transport of that iRNA within the host to impart a therapeutic and/or prophylactic effect by inhibiting function of the target nucleotide sequence. The methodology of the present invention may be practiced by the artisan with any iRNA agent possessing the ability to down-modulate expression of the target gene, including but not limited to any iRNA agent with the ability to therapeutically control expression of a mutant htt gene associated with HD symptoms. It will be known to the artisan that one aspect of practicing the present invention will be the use of an iRNA agent conjugated to a lipophilic agent. The iRNA agent has an antisense strand complementary to a nucleotide sequence of the target nucleic acid, and a sense strand sufficiently complementary to hybridize to the antisense strand.

The iRNA agent may include a liphophilic moiety that facilitates its uptake into a neuron. In one embodiment, the lipophilic moiety is a cholesterol.

In another embodiment, the iRNA agent includes a modification that improves the stability or distribution of the iRNA agent in a biological sample.

The iRNA agents can further be in isolated form or can be part of a pharmaceutical composition used for the methods described herein, particularly as a pharmaceutical composition formulated for delivery to a neuron or formulated for parental administration. The pharmaceutical compositions can contain one or more iRNA agents, and in some embodiments, will contain two or more iRNA agents. In one embodiment, the iRNA agent includes a 2′-modified nucleotide, e.g., a 2′-O-methylated nucleotide. In another embodiment, the iRNA agent includes a phosphorothioate. In another embodiment, the iRNA agent targets a wildtype nucleic acid, e.g., a wildtype htt RNA, involved in the pathogenesis of a neurological disorder, and in yet another embodiment, the iRNA agent targets a polymorphism or mutation of the nucleic acid. In certain embodiments, the iRNA agent can target a sequence in a codon of the open reading frame, the 3′UTR or the 5′UTR of the mRNA transcript of the gene involved in the neurological disorder. In one embodiment, the iRNA agent targets a spliced isoform of mRNA. In another embodiment, the human carries a form of the huntingtin gene that includes an expanded CAG trinucleotide repeat, i.e., more than 30 CAG trinucleotide repeats (e.g., 35, 40, 50, 60, 70, 80, 90, 100 or more CAG trinucleotide repeats), which results in an abnormal form of the huntingtin polypeptide including an expansion of the polypeptide's normal polyglutamine tract. In another embodiment, the human is diagnosed with Huntington's Disease (HD). In one embodiment, the human carries a polymorphism or mutation in the huntingtin gene. For example, the human can carry a polymorphism at position 171, e.g., an A171C polymorphism, in the huntingtin gene according to the sequence numbering in GenBank Accession No. NM_{—}002111 (Aug. 8, 2005). In another embodiment, the iRNA agent targets a nucleic acid that encodes a polypeptide known to interact with the huntingtin protein. For example, the iRNA agent can target a Huntington-associated protein-1 (HAP-1) nucleic acid. In yet another embodiment, the methods disclosed herein may utilize an iRNA agent modified for enhanced uptake into neurons, e.g., conjugated to a cholesterol, which is at least 21 nucleotides long and includes a sense RNA strand and an antisense RNA strand, wherein the antisense RNA strand is 25 or fewer nucleotides in length, and the duplex region of the iRNA agent is 18-25 nucleotides in length. The iRNA agent may further include a nucleotide overhang having 1 to 4 unpaired nucleotides, and the unpaired nucleotides may have at least one phosphorothioate dinucleotide linkage. The nucleotide overhang can be, e.g., at the 3′ end of the antisense strand of the iRNA agent.

Therefore, the present invention relates to a method of downregulating expression of a target gene in a neuron which includes contacting and administering locally an iRNA agent with the neuron for a time sufficient to allow uptake of the iRNA agent into the cell, followed by retrograde transport of the iRNA agent to maximize the therapeutic or prophylactic effect to additional regions of the CNS. As discussed above, the iRNA agent includes a sense strand and an antisense strand that form an RNA duplex. The iRNA agent may also comprise a lipophilic moiety, e.g., a cholesterol, and the antisense strand of the iRNA agent comprises a nucleotide sequence sufficiently complementary to a target sequence of about 18 to 25 nucleotides of an RNA expressed from the target gene. In one embodiment, the lipophilic moiety is conjugated to at least one end of the sense strand, e.g., to the 3′ end of the sense strand. In another embodiment, the sense strand and the antisense strand have a sequence selected from the sense and antisense strands listed in Table 1.

The present invention also relates to a method of treating a human that includes identifying a human diagnosed as having or at risk for developing a neurological disorder, and administering to the human an iRNA agent that targets a gene expressed in a neuron and imparts an improved therapeutic activity by being transported to additional regions, in a retrograde fashion, within the CNS so as to downregulate the target gene in neurons whose cell bodies are located away from the site of local administration. In one embodiment, expression of the gene is associated with symptoms of the neurological disorder. In another embodiment, the iRNA agent includes a sense strand and an antisense strand that form an RNA duplex, and the iRNA agent optionally includes a lipophilic moiety, e.g., a cholesterol. In another embodiment, the antisense strand of the iRNA agent includes a nucleotide sequence sufficiently complementary to a target sequence of about 18 to 25 nucleotides of an RNA expressed from the target gene. In another embodiment, the lipophilic moiety is conjugated to at least one end of the sense strand, e.g., to the 3′ end of the sense strand, and in another embodiment, the iRNA agent includes a phosphorothioate or a 2′ modification, e.g., a 2′OMe or 2′O-fluoro modification. In one embodiment, the sense and antisense strands include a sequence selected from the sense and antisense strands listed in Table 1. Examples of antisense sequences are provided in Table 1 as a guide, and not a limitation, of such sequences. One aspect of the invention provides for utilizing such antisense strand seqeunces as listed in Table 1, or such sequences which differ from an antisense strand listed in Table 1 by no more than 1, 2, 3, 4, or 5 nucleotides. Another aspect of the invention provides for utilizing a sense strand of an iRNA agent optionally conjugated to a lipophilic agent that has the sequence of an antisense strand listed in Table 1, or differs from an antisense strand listed in Table 1 by no more than 1, 2, 3, 4, or 5 nucleotides. Additionally, the antisense strand of the iRNA agent may optionally have at least one modification described in Table 1 or Table 2 (e.g., a cholesterol, 2′-OMe, phosphorothioate, or Cy-3 modification). Also, the antisense strand may have the modifications shown in Table 1 or Table 2. The antisense strand of an iRNA agent can have one or fewer modifications, e.g., the type shown in Table 1 or Table 2, or can have one or more additional modifications, e.g., the type shown in Table 1 or Table 2. In addition, the sense strand of the iRNA agent may have at least one modification described in Table 1 or Table 2 (e.g., a cholesterol, 2′-OMe, phosphorothioate, or Cy-3 modification) and/or may have the modifications shown in Table 1 or Table 2. The sense strand of an iRNA agent can have one or fewer modifications, e.g., the type shown in Table 1 or Table 2, or can have one or more additional modifications, e.g., the type shown in Table 1 or Table 2. To this end, the HD treatment disclosed herein will utilize an iRNA agent that targets an htt nucleic acid, including but not limited to an iRNA agent having an antisense sequence described herein, e.g., an antisense sequence listed in Table 1. In another embodiment for practicing the present invention, the sense strand of the iRNA agent includes the nucleotide sequence of a sense strand described herein, e.g., a sense sequence listed in Table 1. In yet another embodiment, the antisense strand of the iRNA agent overlaps an antisense sequence described herein, e.g., an antisense sequence listed in Table 1, e.g., by at least 1, 5, 10, 11, 12, 13, 14, 15, 16, 17, 18, 19, 20, 21, 22, 23, or 24 nucleotides. Likewise, the sense strand of the iRNA agent overlaps a sense sequence described herein, e.g., a sense sequence listed in Table 1, e.g., by at least 1, 5, 10, 11, 12, 13, 14, 15, 16, 17, 18, 19, 20, 21, 22, 23, or 24 nucleotides.

In another embodiment, the sense strand of the iRNA agent can include at least one mismatch within the antisense strand of the oligonucleotide agent. The mismatch can confer an advantage on the iRNA agent, such as by enhancing antisense strand selection by the RNAi Induced Silencing Complex (RISC). In one embodiment, the mismatch is at least 1, 2, 3, 4, or 5 nucleotides away from the 3′-terminal nucleotide of the sense strand. In another embodiment, the RNA agent includes an antisense strand that is substantially complementary to a sequence encoded by a region of the human htt gene including or overlapping a sequence provided in GenBank Accession Number NM_{—}002111 (Aug. 8, 2005). In certain embodiments, the iRNA agents can target an htt RNA and can include a sense and/or antisense sequence listed in Table 1. In additional embodiments regarding the methodology disclosed herein, the iRNA agent includes at least one modification in addition to the lipophilic moiety for enhanced uptake into neurons. The at least one additional modification can be, e.g., a phosphorothioate or 2′O-methyl (2′OMe) modification.

TABLE 1

iRNA Agents Targeting htt

| AL-DP- | | |
|---|---|---|
| Number | sense: 5′-3′ | antisense: 5′-3′ |
| AL-DP- | Cy3cuG cuu uAG ucG AGA Acc | UGG UUC UCG ACu |
| 4630 | ATsT | AAA GcA GTsT |
| AL-DP- | Cy3cuG cuu uAG ucG AGA Acc | UGG UUC UCG ACu |
| 4631 | ATTsChol | AAA GcA GTsT |

Note:
capital letters represent unmodified bases, small letters represent 2′-O-methyladenosine-5′-phosphate modifications, ‘s’ represents a phosphorothioate bound inbetween neighboring bases, ‘Chol’ represents cholesterol-conjugate, ‘Cy3’ stands for a Cy3 conjugate

TABLE 2
Oligonucleotide Ligand Conjugates and Ligand Building Blocks

Oligonucleotide ligand conjugates

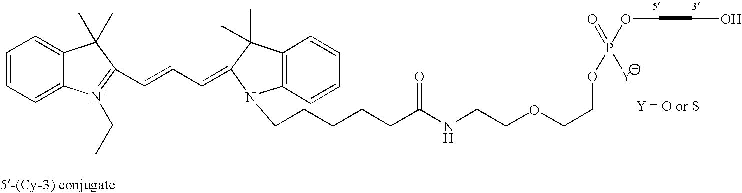

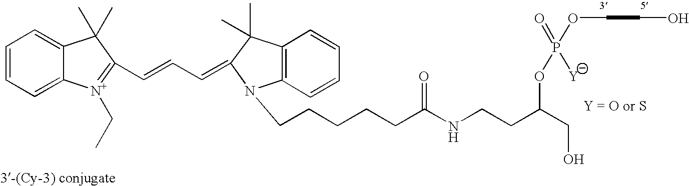

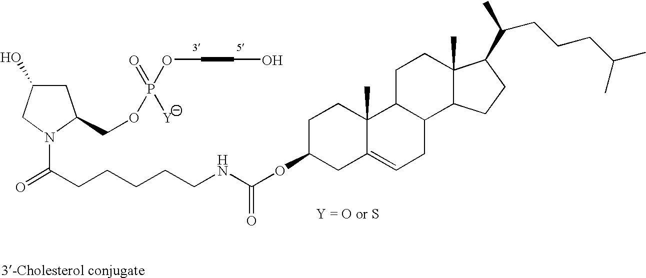

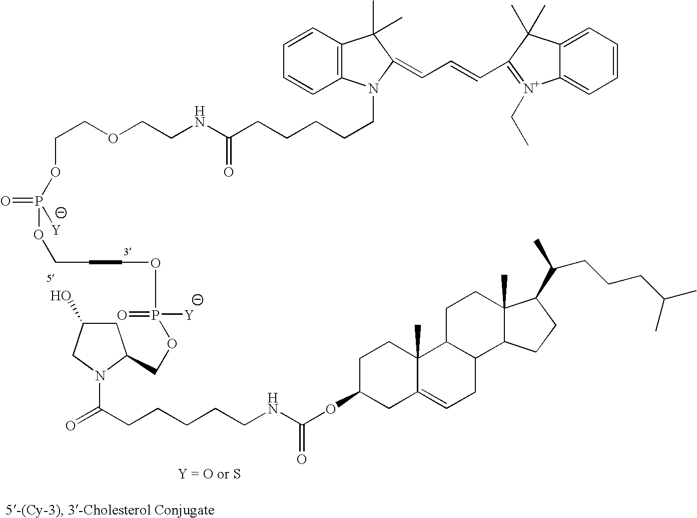

Ligand building blocks

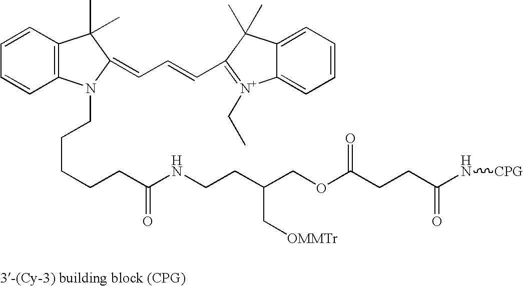

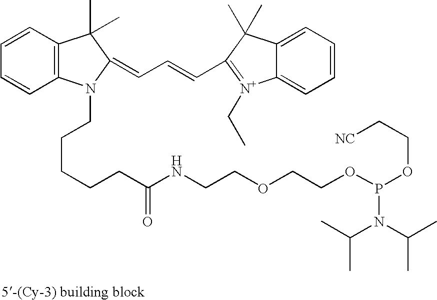

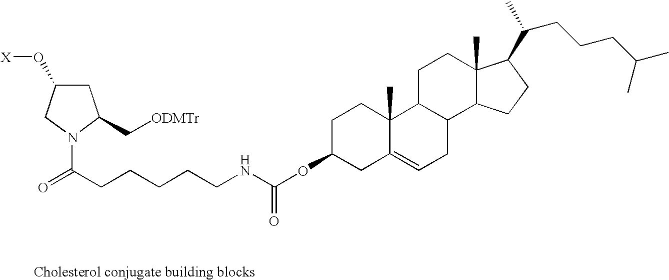

The present invention also relates to methods disclosed herein which feature a pharmaceutical composition including an iRNA agent optionally conjugated to a lipophilic moiety for enhanced uptake into neurons, e.g., conjugated to a cholesterol molecule, and a pharmaceutically acceptable carrier. The iRNA agent targets a nucleic acid involved in a neurological disease or disorder. In a specific embodiment, the pharmaceutical composition utilized in the disclosed methods includes an iRNA agent targeting an htt nucleic acid and a pharmaceutically acceptable carrier. The iRNA agent has an antisense strand complementary to a nucleotide sequence of an htt RNA, and a sense strand sufficiently complementary to hybridize to the antisense strand. In one embodiment, the iRNA agent includes a lipophilic moiety that facilitates its uptake into a neuron. In one embodiment, the lipophilic moiety is a ligand that includes a cationic group. In another embodiment, the lipophilic moiety is attached to one or both ends of one or both strands of the iRNA agent. In a yet another embodiment, the lipophilic moiety is attached to one end of the sense strand of the iRNA agent, and in yet another embodiment, the ligand is attached to the 3′ end of the sense strand. In certain embodiments, the lipophilic agent is, e.g, cholesterol, vitamin E, vitamin K, vitamin A, folic acid or a cationic dye, such as Cy3. In a preferred embodiment, the lipophilic moiety is a cholesterol.

In another embodiment, the iRNA agent of the pharmaceutical composition may also include a modification that improves the stability or distribution of the iRNA agent in a biological sample. The iRNA agents can further be in isolated form or can be part of a pharmaceutical composition used for the methods described herein, particularly as a pharmaceutical composition formulated for delivery to a neuron or formulated for parental administration. The pharmaceutical compositions can contain one or more iRNA agents, and in some embodiments, will contain two or more iRNA agents. In one embodiment, the iRNA agent includes a 2′-modified nucleotide, e.g., a 2′-O-methylated nucleotide. In another embodiment the iRNA agent includes a phosphorothioate.

In another embodiment, htt RNA levels in a neuron are reduced by contacting the neuron of the subject with an iRNA agent which may optionally be modified for enhanced uptake into neurons. In a preferred embodiment, the iRNA agent is modified with a lipophilic moiety such as cholesterol. Therefore, practice of the present invention discloses relies on generating an iRNA agent that targets a nucleic acid expressed in neurons and that is modified for enhanced uptake into neurons. The method includes selecting a nucleotide sequence of between 18 and 25 nucleotides long from the nucleotide sequence of a target mRNA, e.g., an htt mRNA, and synthesizing the iRNA agent. The sense strand of the iRNA agent includes the nucleotide sequence selected from the target RNA, and the antisense strand is sufficiently complementary to hybridize to the sense strand. In one embodiment, the iRNA agent is unconjugated. In another embodiment, the method includes incorporating at least one lipophilic moiety into the iRNA agent, e.g., onto at least one end of the sense strand of the iRNA agent. Additionally, the lipophilic moiety may be incorporated onto the 3′ end of the sense strand of the iRNA agent. In one embodiment, a cationic dye, e.g., Cy3, is incorporated into at least one strand of the iRNA agent, e.g., on the 3′ or 5′ end of the iRNA agent. In one embodiment, more than one lipophilic moiety, e.g., more than one different kind of lipophilic moiety is incorporated into the iRNA agent. In certain embodiments, the iRNA agent includes the ligand conjugates illustrated in Table 1 or Table 2. In other embodiments the method of making the iRNA agent includes use of the building blocks illustrated in Table 1 or Table 2. In yet other embodiments, the methods featured in the invention include the iRNA agents listed in Table 1 or Table 2, which target htt RNA. In one embodiment, the method further includes administering the iRNA agent to a subject, e.g., a mammalian subject, such as a human subject, such as a human having or at risk for developing a neurological disease or disorder. In one embodiment, the human has or is at risk for developing HD.

The methods and compositions featured in the invention, e.g., the methods and iRNA compositions to treat the neurological disorders described herein, can be used with any dosage and/or formulation described herein, as well as with any route of administration described herein. A neurological disease or disorder is any disease or disorder that affects the nervous system (the central or peripheral nervous system). Exemplary neurological diseases and disorders include Huntingtons's Disease (HD), Parkinson's Disease (PD), Amyotropic Lateral Sclerosis (ALS), Alzheimer's Disease, Lewy body dementia, Multiple System Atrophy, spinal and bulbar muscular atrophy (Kennedy's disease), Tourette Syndrome, Autosomal dominant spinocerebellar ataxia (SCA) (e.g., Type 1 SCA1, Type 2 SCA2, Type 3 (Machado-Joseph disease) SCA3/MJD, Type 6 SCA6, Type 7 SCA7, Type 8 SCA8, Friedreich's Ataxia and Dentatorubral pallidoluysian atrophy DRPLA/Haw-River syndrome), schizophrenia, age associated memory impairment, autism, attention-deficit disorder, and bipolar disorder.

Any patient having a neurological disease or disorder is a candidate for treatment with a method or composition described herein. Presymptomatic subjects can also be candidates for treatment with an iRNA agent targeted to neurons. For example, a presymptomatic human determined to be at risk for HD is a candidate for treatment with an anti-htt iRNA agent conjugated to a lipophilic molecule, e.g., a cholesterol molecule, for delivery to neurons. In one embodiment, a presymptomatic candidate is identified by either or both of risk-factor profiling, such as, for example, genetic screening, and functional neuroimaging (e.g., by fluorodopa and positron emission tomography). For example, the candidate subject can be identified by risk-factor profiling followed by functional neuroimaging.

Individuals having a particular genotype are candidates for treatment. In some embodiments the patient will carry a particular genetic mutation that places the patient at increased risk for developing a disorder of the nervous system, e.g., HD. For example, an individual carrying a CAG trinucleotide expansion in the htt gene (e.g., more than 36 repeats) is at increased risk for developing HD and is a candidate for treatment with an iRNA agent featured in the invention, e.g., conjugated to a cholesterol molecule for enhanced uptake into neurons. The iRNA agent preferably targets the htt gene. In addition, a SNP in the htt gene has been found to be an indicator of the presence of the expanded CAG repeat that triggers HD. The SNP is an A to C polymorphism at position 171, according to the numbering of GenBank Accession No. NM_{—}002111. A human carrying this SNP is therefore a candidate for treatment with an iRNA agent featured in the invention, or is at least a candidate for further genetic studies (such as for testing for the CAG repeat expansion) which will further determine if the human is a candidate for treatment with an iRNA agent targeting htt and modified for enhanced delivery to neurons. Candidate iRNA agents can be designed by performing, for example, a gene walk analysis. Overlapping, adjacent, or closely spaced candidate agents corresponding to all or some of the transcribed region can be generated and tested. Each of the iRNA agents can be tested and evaluated for the ability to down regulate target gene expression, as disclosed below.

An iRNA agent (such as a ds siRNA) for use in the disclosed methods can be rationally designed based on sequence information and desired characteristics. For example, an iRNA agent can be designed according to the relative melting temperature of the candidate duplex. Generally, the duplex will have a lower melting temperature at the 5′ end of the antisense strand than at the 3′ end of the antisense strand.

The iRNA agent can be coupled, e.g., covalently coupled, to a second agent. For example, an iRNA agent used to treat a particular neurological disorder can be coupled to a second therapeutic agent, e.g., an agent other than the iRNA agent. The second therapeutic agent can be one which is directed to the treatment of the same neurological disorder. For example, in the case of an iRNA used to treat a HD, the iRNA agent can be coupled to a second agent which is known to be useful for the treatment of HD. The iRNA agents described herein can be formulated for administration to a subject. In another embodiment, an iRNA preparation can be formulated in combination with another agent, e.g., another therapeutic agent or an agent that stabilizes an iRNA, e.g., a protein that complexes with iRNA to form an iRNP. Still other agents include chelators, e.g., EDTA (e.g., to remove divalent cations such as Mg2+), salts, RNAse inhibitors (e.g., a broad specificity RNAse inhibitor such as RNAsin) and so forth.

In another aspect of the invention, antigen can be used to target an iRNA to a neuron in the brain. In one embodiment, the targeting moiety is attached to a liposome. For example, U.S. Pat. No. 6,245,427 describes a method for targeting a liposome using a protein or peptide. In another example, a cationic lipid component of the liposome is derivatized with a targeting moiety. For example, WO 96/37194 describes converting N-glutaryldioleoylphosphatidyl ethanolamine to an N-hydroxysuccinimide activated ester. The product was then coupled to an RGD peptide.

A composition that includes an iRNA agent targeting a gene expressed in neurons can be delivered to a subject by a variety of routes. Exemplary routes include intrastriatal, intracerebroventricular, intrathecal, intraparenchymal (e.g., in the striatum), nasal, and ocular delivery. The composition can also be delivered systemically, e.g., by intravenous, subcutaneous or intramuscular injection, which is particularly useful for delivery of the iRNA agents to peripheral neurons. A preferred route of delivery is directly to the brain, e.g., into the ventricles or the hypothalamus of the brain, or into the lateral or dorsal areas of the brain. The iRNA agents for neuronal delivery can be incorporated into pharmaceutical compositions suitable for administration. For example, compositions can include one or more species of an iRNA agent and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. A pharmaceutically acceptable carrier does not include a transfection reagent or a reagent to facilitate uptake in a neuron that is in addition to the lipophilic moiety conjugated to the iRNA agent featured in the invention. The use of such media and agents for pharmaceutically active substances is well known in the art. Except insofar as any conventional media or agent is incompatible with the active compound, use thereof in the compositions is contemplated. Supplementary active compounds can also be incorporated into the compositions. In one embodiment, the iRNA agent can be delivered by way of a cannula or other delivery device having one end implanted in a tissue, e.g., the brain, e.g., the striatum, substantia nigra, cortex, hippocampus, or globus pallidus of the brain. The cannula can be connected to a reservoir of iRNA agent. The flow of delivery can be mediated by a pump, such as any implantable pump device known in the art which allows for regulated delivery of the iRNA agent throughout the treatment course. Any such pump may be utilized to practice this aspect of the invention, including but not limited to a drug reservoir and/or a drug pump of any kind, for example an osmotic pump, an infusion pump, an electromechanical pump, an electroosmotic pump, an effervescent pump, a hydraulic pump, a piezoelectric pump, an elastomeric pump, a vapor pressure pump, or an electrolytic pump. Preferably, such a pump is implanted within the body. The flow or delivery of the iRNA agent can be mediated by the pump. Both osmotic and infusion pumps are commercially available from a variety of suppliers, including but not limited to a SynchroMed pump (Medtronic, Minneapolis, Minn.). In one embodiment, a SynchroMed pump and reservoir are implanted in an area distant from the tissue, e.g., in the abdomen, and delivery is effected by a conduit leading from the pump or reservoir to the site of release. Devices for delivery to the brain are described, for example, in U.S. Pat. Nos. 6,093,180, and 5,814,014 and are recently reviewed by Misra, et al. (2003 J. Pharm. Parmaceut. Sci. 6(2):252-273. In view of the teachings herein, one of skill in the art can readily determine which general area of the CNS is an appropriate target. As exemplified herein, the striatum is a suitable area of the brain to target an iRNA agent. Stereotactic maps and positioning devices are available and positioning may be effected by the use of anatomical maps obtained by CT and/or MRI imaging of the subject's brain to help guide the injection device to the chosen target. A therapeutic or prophylactic amount effective to treat a CNS disorder by the methods disclosed herein will comprise a sufficient amount of the iRNA agent during the entire course of treatment so as to ameliorate or reduce the symptoms of the CNS disorder being targeted for treatment. As noted herein, these iRNA agents may also contain a pharmaceutically acceptable carrier or excipient. Such carriers or excipients include any pharmaceutical agent that does not itself induce the production of antibodies harmful to the individual receiving the composition, and which may be administered without undue toxicity.

The route of delivery can be dependent on the disorder of the patient. For example, a subject diagnosed with HD can be administered an anti-htt iRNA agent, which optionally may be conjugated to a lipophilic agent, directly into the brain (e.g., into the globus pallidus or the corpus striatum of the basal ganglia, and near the medium spiny neurons of the corpus striatum). For the treatment of HD, for example, symptomatic therapies can include the drugs haloperidol, carbamazepine, or valproate. Other therapies can include psychotherapy, physiotherapy, speech therapy, communicative and memory aids, social support services, and dietary advice. A pharmaceutical composition containing an iRNA agent can be delivered to the patient by injection directly into the area containing the disease-affected cells. For example, the pharmaceutical composition can be delivered by injection directly into the brain. The injection can be by stereotactic injection into a particular region of the brain (e.g., the substantia nigra, cortex, hippocampus, striatum, or globus pallidus). The iRNA agent can be delivered into multiple regions of the central nervous system (e.g., into multiple regions of the brain, and/or into the spinal cord). The iRNA agent can be delivered into diffuse regions of the brain (e.g., diffuse delivery to the cortex of the brain).

A pharmaceutical composition containing an iRNA agent either in an unconjugated form or conjugated to a lipophilic moiety for enhanced uptake into neurons can be administered to any patient diagnosed as having or at risk for developing a neurological disorder, such as HD. In one embodiment, the patient is diagnosed as having a neurological disorder, and the patient is otherwise in general good health. For example, the patient is not terminally ill, and the patient is likely to live at least 2, 3, 5, or 10 years or longer following diagnosis. The patient can be treated immediately following diagnosis, or treatment can be delayed until the patient is experiencing more debilitating symptoms. In general, an iRNA agent can be administered by any suitable method. As used herein, topical delivery can refer to the direct application of an iRNA agent to any surface of the body, including the eye, a mucous membrane, surfaces of a body cavity, or to any internal surface. Formulations for topical administration may include transdermal patches, ointments, lotions, creams, gels, drops, sprays, and liquids. Conventional pharmaceutical carriers, aqueous, powder or oily bases, thickeners and the like may be necessary or desirable. Topical administration can also be used as a means to selectively deliver the iRNA agent to the epidermis or dermis of a subject, or to specific strata thereof, or to an underlying tissue.

Compositions for intrastriatal, intrathecal or intraventricular (e.g., intracerebroventricular) administration may include sterile aqueous solutions which may also contain buffers, diluents and other suitable additives. Compositions for intrastriatal, intrathecal or intraventricular administration preferably do not include a transfection reagent or an additional lipophilic moiety besides the lipophilic moiety attached to the iRNA agent. Formulations for parenteral administration may include sterile aqueous solutions which may also contain buffers, diluents and other suitable additives. Intrastriatal or intraventricular injection may be facilitated by a catheter, for example, attached to a reservoir, as discussed above. Preferably, the total concentration of solutes should be controlled to render the preparation isotonic.

The term “therapeutically effective amount” and/or “prophylactically effective amount” is the amount present in the composition that is needed to provide the desired level of drug in the subject to be treated to give the anticipated physiological response.

The term “physiologically effective amount” is that amount delivered to a subject to give the desired palliative or curative effect.

The term “pharmaceutically acceptable carrier” means that the carrier has no significant adverse toxicological effects.

The types of pharmaceutical excipients that are useful as carrier include stabilizers such as human serum albumin (HSA), bulking agents such as carbohydrates, amino acids and polypeptides; pH adjusters or buffers; salts such as sodium chloride; and the like. These carriers may be in a crystalline or amorphous form or may be a mixture of the two.

Suitable pH adjusters or buffers include organic salts prepared from organic acids and bases, such as sodium citrate, sodium ascorbate, and the like; sodium citrate is preferred.

An iRNA agent can be administered by oral or nasal delivery. For example, drugs administered through these membranes have a rapid onset of action, provide therapeutic plasma levels, avoid first pass effect of hepatic metabolism, and avoid exposure of the drug to the hostile gastrointestinal (GI) environment. Additional advantages include easy access to the membrane sites so that the drug can be applied, localized and removed easily. In one embodiment, an iRNA agent administered by oral or nasal delivery has been modified to be capable of traversing the blood-brain barrier.

In one embodiment, unit doses or measured doses of a composition that include iRNA are dispensed by an implanted device. The device can include a sensor that monitors a parameter within a subject. For example, the device can include a pump, such as an osmotic pump and, optionally, associated electronics.

In one embodiment, the iRNA agent pharmaceutical composition includes a plurality of iRNA agent species. In another embodiment, the iRNA agent species has sequences that are non-overlapping and non-adjacent to another species with respect to a naturally occurring target sequence. In another embodiment, the plurality of iRNA agent species is specific for different naturally occurring target genes.

In certain other aspects, the invention provides kits that include a suitable container containing a pharmaceutical formulation of an iRNA agent, e.g., a double-stranded iRNA agent, or sRNA agent, (e.g., a precursor, e.g., a larger iRNA agent which can be processed into a sRNA agent, or a DNA which encodes an iRNA agent, e.g., a double-stranded iRNA agent, or sRNA agent, or precursor thereof). In certain embodiments the individual components of the pharmaceutical formulation may be provided in one container. Alternatively, it may be desirable to provide the components of the pharmaceutical formulation separately in two or more containers, e.g., one container for an iRNA agent preparation, and at least another for a carrier compound. The kit may be packaged in a number of different configurations such as one or more containers in a single box. The different components can be combined, e.g., according to instructions provided with the kit. The components can be combined according to a method described herein, e.g., to prepare and administer a pharmaceutical composition. The kit can also include a delivery device.

Specific embodiments according to the methods of the present invention will now be described in the following examples. Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

### EXAMPLES

Animal surgery and dosing of test articles (Charles River Study VSX00021) was performed by Charles River Laboratory in accordance with their Standard Operating Protocol. All surgeries were done under aseptic conditions. The surgical site was prepared for aseptic surgery by wiping the area with Betadyne® (10% povidone iodine; Purdue Frederick Company, Stamford, Conn.) scrub solution to remove all detritus, followed by wiping the area with sponges soaked in 70% isopropyl alcohol which were allowed to dry. Eighteen (18) male Sprague Dawley rats with body weights of approximately 350 grams each were surgically and stereotaxic implanted with unilateral intrastriatal cannulas (stereotaxic coordinates were Anteroposterior: +1.0 mm, Mediolateral relative to bregma: 2.5 mm and Dorsoventral: 5 mm) under anesthesia and aseptic conditions. Each rat received an intraperitoneal (IP) injection of ketamine (87 mg/kg) and xylazine (13 mg/kg) for anesthesia. Prior to full recovery from anesthesia, the animals were in some cases given an injection of buprenorphine at 0.01 mg/kg subcutaneous (SC) The rats were randomized by body weights into four groups. The 2 groups to receive Cy3-Htt siRNA or Cy3-cholesterol-siRNA consisted of six rats each, whereas the control group to receive phosphate buffered saline consisted of three rats. Twelve days after cannulation, rats were anesthetized and received a SC implant of Alzet mini-osmotic pump 1002 (two weeks capacity at a delivery rate of 0.25 μL/hr) that was then connected to the catheter. Pumps were primed in sterile 0.9% saline at 37° C. for at least four to six hours prior to implantation with the appropriate test article. After 12 days of test article infusion, rats were perfused first with Phosphate Buffered Saline (PBS) followed by perfusion with Fixation solution (specified by Neuroscience Associates—NSA); brains were then collected and placed in fixative overnight. The next day, the brains were transferred to PBS. These brains were then shipped to Neuroscience Associates for sectioning and histological processing according to NSA's Standard Operating Protocol. A maximum of sixteen 40 μm thick individual brain sections were mounted on one slide. Sections were stained with GFAP and Iba1 by NSA. Evaluation of processed sections was carried out at Alnylam. siRNAs were designed and synthesized by Alnylam. The parent sequence for the Cy3-Htt and Cy3-chol-Htt siRNAs was AL-DP-6003. Cy3-Htt siRNA (AL-DP-4630) and Cy3-chol-Htt siRNA (AL-DP-4631) duplexes (Table 3) were annealed in 1× PBS at a final concentration of 2 mM.

TABLE 3

Sequences of Cy3-tagged siRNAs AL-DP-4630 and
AL-DP-4631

| AL-DP- | | |
|---|---|---|
| Number | sense: 5′-3′ | antisense: 5′-3′ |
| AL-DP- | Cy3cuG cuu uAG ucG AGA Acc | UGG UUC UCG ACu |
| 4630 | ATsT | AAA GcA GTsT |
| AL-DP- | Cy3cuG cuu uAG ucG AGA Acc | UGG UUC UCG ACu |
| 4631 | ATTsChol | AAA GcA GTsT |

Note:
capital letters represent unmodified bases, small letters represent 2′-O-methyladenosine-5′-phosphate modifications, ‘s’ represents a phosphorothioate bound inbetween neighboring bases, ‘Chol’ represents cholesterol-conjugate, ‘Cy3’ stands for a Cy3 conjugate

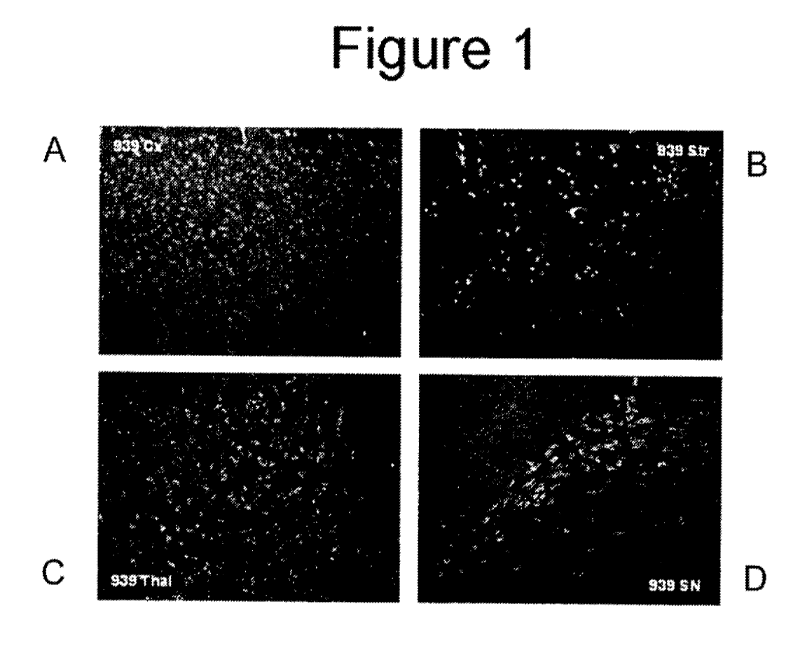

As expected, there were no fluorescent signals observed in PBS control brains. The distribution profile of the unconjugated Cy3-Htt siRNA after infusion with 180 μg per day for 12 days showed distinct neuronal uptake in cortex, striatum, thalamus and substantia nigra (FIG. 1). The distance of the Cy3-Htt siRNA uptake was about 3.5 mm from the frontal cortex to the medial striatum (Interaural 12.70 mm to 9.20 mm, Paxinos and Watson) and it extended to the thalamus and substantia nigra, in a pattern consistent with retrograde transport of siRNA, rather than diffusion to these structures. Brain regions other than thalamus and substantia nigra, although located at a similar distance from the injection site, did not contain detectable Cy3-Htt siRNA.

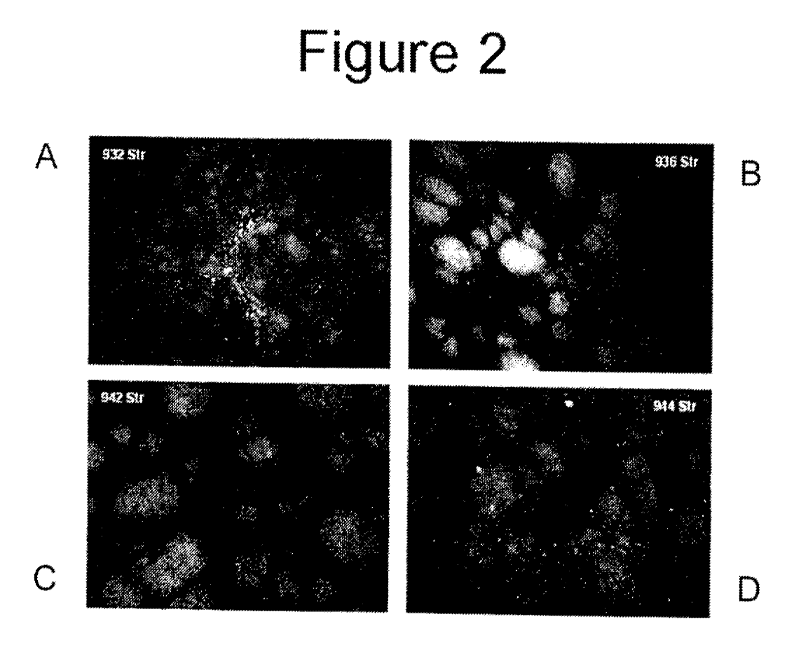

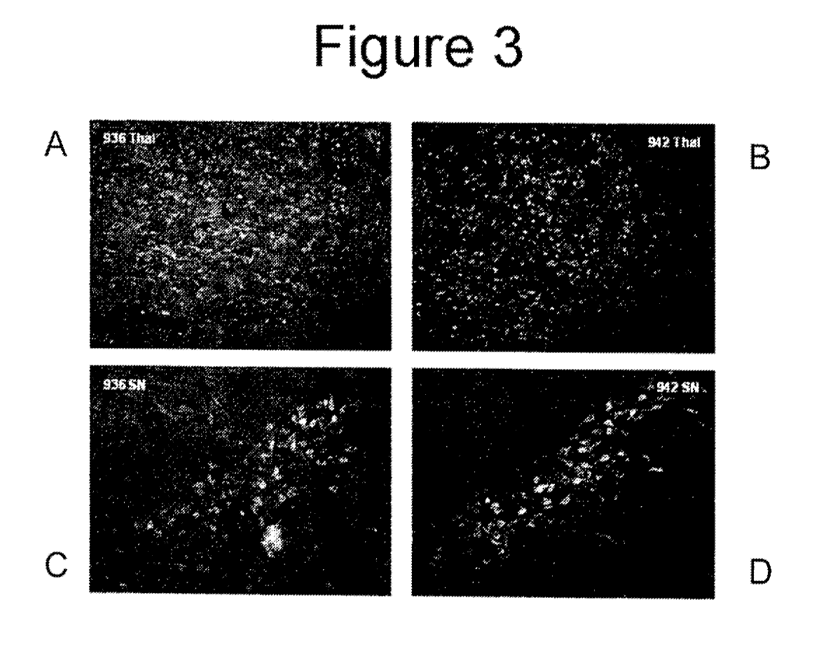

The distribution pattern of cholesterol-conjugated Cy3-Htt siRNA was similar to unconjugated Cy3-Htt siRNA but with much higher intensity in cortex and around the infusion site of the striatum. Most of the uptake in the cortex and striatum appeared to be within fiber tracks or neuronal processes (FIG. 2). After infusion with 180 μg cholesterol-conjugated Cy3-Htt siRNA per day for 12 days, neuronal labeling was present in the thalamus and substantia nigra (FIG. 3).

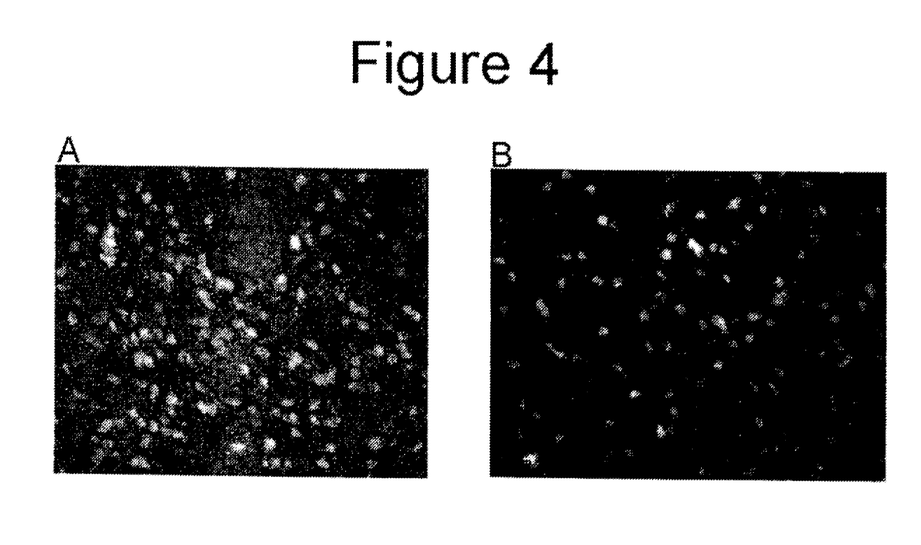

Consistent with the neuronal morphology of labeled cells, there was no overlap of Cy3 with Iba1- and GFAP-immunoreactivity. These results demonstrate neuronal uptake after infusion of unconjugated and cholesterol-conjugated Cy3-Htt siRNAs (FIG. 4A and 4B).

The same regions of the brain-cortex, striatum, thalamus and substantia nigra- were labeled after a single bolus injection of Cy3-tagged siRNA, although much broader labeling in cells of neuronal morphology was present overall within these regions after osmotic pump infusion than after a single bolus injection. Nonetheless, the distribution pattern after a single bolus injection of Cy3-tagged siRNA suggests that retrograde transport of siRNA can occur after a single bolus injection as well as after osmotic pump infusion over longer periods of time.

Endothelial cells or pericytes were also labeled after both unconjugated and cholesterol-conjugated Cy3-Htt siRNA infusion.

The data within this Example section show that (i) cortical, striatal, thalamic and substantia nigra neurons can be targeted by siRNA (unconjugated and cholesterol conjugated) formulated in PBS via intrastriatal pump infusion, as well as after a single bolus injection; (ii) intrastriatal pump infusion may provide broad neuronal delivery of siRNA targeting the htt gene, via retrograde neuronal transport from the site of siRNA administration to other regions of the brain; (iii) fiber tracts in striatum can be targeted by cholesterol-conjugated siRNA formulated in PBS with intrastriatal pump infusion; (iv) pericytes around capillaries can be targeted by siRNA (unconjugated and cholesterol conjugated) via intrastriatal pump infusion. These results indicate that intrastriatal siRNA infusion via an osmotic mini pump can result in widespread distribution of siRNA in the brain via retrograde transport. Therefore, siRNA infusion into the CNS represents a treatment strategy for Huntington's disease that may provide broad neuronal effects in regions at or near the site of infusion as well as in regions distant from the site of infusion that are anatomically connected by neuronal pathways.

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

What is claimed is:

1. A method of treating a central nervous system disorder in a mammal which comprises administering a composition to a neural cell at a first site within the central nervous system, wherein the composition comprises an iRNA agent with an antisense sequence that is substantially complementary to a target RNA in the neural cell such that the iRNA agent decreases expression of the target RNA in the neural cell of the mammal, and wherein the iRNA agent undergoes retrograde transport from the first site to one or more secondary sites within the central nervous system to act in a therapeutically effective manner away from the first site and where the distance between the first and a second site is at least 2 mm.

2. The method of claim 1 wherein the mammal is a human.

3. The method of claim 2 wherein the central nervous system disorder is associated with or treatable through a suppression of the target RNA.

4. The method of claim 3 wherein the disorder is selected from the group consisting of Alzheimer's disease, Parkinson's disease, Huntington's disease, spinocerebellar ataxia 1, 2, 3, 6, 7, and 17, dentarubral-pallidoluysian atrophy, spinobulbar muscular atrophy, myotonic dystrophy and motor neuron disorders.

5. The method of claim 4 wherein the dominantly inherited disease is Huntington's disease and the target RNA is a huntingtin RNA.

6. The method of claim 5 wherein the iRNA agent is a double stranded RNA duplex.

7. The method of claim 6 wherein the iRNA agent further comprises a lipophilic moiety.

8. The method of claim 7 wherein the lipophilic moiety is a cholesterol.

9. The method of claim 5 wherein the antisense sequence differs by no more than four nucleotides from an antisense sequence listed in Table 2.

10. The method of claim 5 wherein the antisense sequence is an antisense sequence listed in Table 2.

11. A method of treating a central nervous system disorder in a mammal which comprises administering a composition to a neural cell at a first site in the central nervous system by intrastriatal infusion, wherein the composition comprises an iRNA agent with an antisense sequence that is substantially complementary to a target RNA in the neural cell such that the iRNA agent decreases expression of the target RNA in a neural cell of the mammal, and wherein the iRNA agent undergoes retrograde transport from the first site to one or more secondary sites within the central nervous system to act in a therapeutically effective manner away from the first site and where the distance between the first and a second site is at least 2 mm.

12. The method of claim 11 wherein the mammal is a human.

13. The method of claim 12 wherein the central nervous system disorder is a dominantly inherited nucleotide repeat disease.

14. The method of claim 13 wherein the secondary sites are selected from the group consisting of the cortex, thalamus, substantial nigra of the central nervous system, or any combination thereof.

15. The method of claim 14 wherein the dominantly inherited nucleotide repeat disease is Huntington's disease and the target RNA is a huntingtin RNA.

16. The method of claim 15 wherein the iRNA agent is a double stranded RNA duplex.

17. The method of claim 16 wherein the iRNA agent further comprises a lipophilic moiety.

18. The method of claim 17 wherein the lipophilic moiety is a cholesterol.

19. The method of claim 15 wherein the antisense sequence differs by no more than four nucleotides from an antisense sequence listed in Table 2.

20. The method of claim 15 wherein the antisense sequence is an antisense sequence listed in Table 2.

21. A method of treating a human in a therapeutic or prophylactic manner which comprises:
a) identifying the human as having or being at risk for developing a central nervous system disorder;
b) administering to a first site of the human an iRNA agent that comprises an antisense sequence which targets a target RNA expressed in a neural cell, such that the iRNA agent undergoes retrograde transport from the first site to one or more secondary sites within the central nervous system to act in a therapeutic or prophylactic manner away from the first site and where the distance between the first and a second site is at least 2 mm.

22. The method of claim 21 wherein the iRNA agent is administered to the first site by interstitial infusion.

23. The method of claim 21, wherein the central nervous system disorder is associated with or treatable through a suppression of the target RNA.

24. The method of claim 23 wherein the disease is selected from the group consisting of Alzheimer's disease, Parkinson's disease, Huntington's disease, spinocerebellar ataxia 1, 2, 3, 6, 7, and 17, dentarubral-pallidoluysian atrophy, spinobulbar muscular atrophy, myotonic dystrophy and motor neuron disorders.

25. The method of claim 24 wherein the dominantly inherited disease is Huntington's disease and the target RNA is a huntingtin RNA.

26. The method of claim 24 wherein the iRNA agent is a double stranded RNA duplex.

27. The method of claim 26 wherein the iRNA agent further comprises a lipophilic moiety.

28. The method of claim 27 wherein the lipophilic moiety is a cholesterol.

29. The method of claim 25 wherein the antisense sequence differs by no more than four nucleotides from an antisense sequence listed in Table 2.

30. The method of claim 25 wherein the antisense sequence is an antisense sequence listed in Table 2.

31. The method of claim 22 wherein the central nervous system disorder is associated with or treatable through a suppression of the target RNA.

32. The method of claim 31 wherein the disease is selected from the group consisting of Alzheimer's disease, Parkinson's disease, Huntington's disease, spinocerebellar ataxia 1, 2, 3, 6, 7, and 17, dentarubral-pallidoluysian atrophy, spinobulbar muscular atrophy, myotonic dystrophy and motor neuron disorders.

33. The method of claim 32 wherein the dominantly inherited disease is Huntington's disease and the target RNA is a huntingtin RNA.

34. The method of claim 33 wherein the iRNA agent is a double stranded RNA duplex.

35. The method of claim 34 wherein the iRNA agent further comprises a lipophilic moiety.

36. The method of claim 35 wherein the lipophilic moiety is a cholesterol.

37. The method of claim 33 wherein the antisense sequence differs by no more than four nucleotides from an antisense sequence listed in Table 2.

38. The method of claim 33 wherein the antisense sequence is an antisense sequence listed in Table 2.
